(19) United States
(12) Patent Application Publication
(10) Pub. No.: US 2003/0153928 A1
(43) Pub. Date: Aug. 14, 2003

(54) Surgical instrument and method for suture ligation

(76) Inventor: Rizk El-Galley, Birmingham, AL (US)

Correspondence Address: BRADLEY ARANT ROSE & WHITE, LLP, INTELLECTUAL PROPERTY DEPARTMENT-NWJ, 1819 FIFTH AVENUE NORTH, BIRMINGHAM, AL, 35203-2104, US

(21) Appl. No.: 10/073,508

(22) Filed: Feb. 11, 2002

(51) Int. Cl. A61B 17/04

(52) U.S. Cl. 606/148

(57) ABSTRACT

The present disclosure describes a modified surgical instrument of conventional design for assisting in suture ligation. The instrument is particularly well suited for suture ligation during endoscopic and laparoscopic procedures, but may be used in conventional surgical procedures as well. The instrument comprises a knot assist assembly operationally coupled to the instrument such that in operation, the knot assist assembly provides the surgeon with a perpendicular tip to aid in suture ligation.

### FIELD OF THE DISCLOSURE

The present disclosure relates generally to an improved method and device for suture ligation during surgical procedures. In particular, the disclosure relates to an improved method and device for suture ligation during laparoscopic and endoscopic procedures.

### BACKGROUND

Suture ligation (meaning forming suture knots during a procedure) is commonly used in almost all surgical techniques. However, due to the nature of laparoscopic surgery, the use of sutures has been somewhat limited as a result of the difficulty of suture ligation. Laparoscopy is a technique for performing a surgical operation on a patient without requiring that a major incision be made. As a result, laparoscopic procedures are considered minimally invasive. The advantages of these minimally invasive procedures include decreased recovery times, decreased hospital stays, decreased pain, decreased infection rates and decreased cost both for the consumer and the health system as a whole. A number of surgical procedures can now be performed via laparoscopy, such as cholecystectomies, acid reflux procedures, removal of small tumors, cardiomyotomy, restriction of the small bowel, lysis of adhesions, electrocoagulation, colectomy, appendectomy, hernia repair, and splenic surgery, just to name a few. As the tools and technology available for laparoscopic procedures becomes more advanced, the list of surgical procedures is bound to grow.

In general, laparoscopic operative technique involves several phases. First, a pneumoperitoneum is established. Second, trocars are inserted into the in the body (through a minor incision) for the introduction of the camera and surgical instruments. A spring-loaded needle with a blunt tip is inserted through one of these small incisions to initiate the pneumoperitoneum. The patient is placed in Trendelenburg's position and insufflation of CO₂ is begun until an intraperitoneal pressure of 12 to 18 mmhg is attained. The needle is then removed and replaced with a trocar through which a videoscope is inserted. After inspecting the peritoneal cavity for trauma caused by the initial procedures, the remaining trocars are inserted under direct laparoscopic observation. The different surgical instruments needed to complete the procedure may then be inserted through the trocars. After the procedure is completed, the trocars are removed and the incisions closed.

During the various laparoscopic procedures, it is often required that body tissue be sutured as a result of the procedure. For example, the procedure might require that a blood vessel be severed, or a blood vessel may be inadvertently cut during the procedure. In addition, the site of the trocar insertion must also be closed. The use of conventional suturing techniques in laparoscopic surgery is consuming and cumbersome. This is due primarily to the fact that the surgeon must form and tie a suture knot inside the body cavity while holding the instruments parallel to the suture thread. As a result, the suture thread often slips off of the laparoscopic instrument, requiring the surgeon to re-initiate the procedure. In conventional surgery, the surgeon is free to place one of the instruments perpendicular to the suture thread in order to form and tie a suture knot. However, because the surgeon's range of motion is restricted in laparoscopic procedures, the surgeon cannot place an instrument perpendicular to the suture thread. This inability to use sutures efficiently in laparoscopic procedures not only increases the time required for the procedure, but also increases the risk of complications resulting from the procedure.

In order to overcome these difficulties, the art has used multiple instruments and techniques. One common technique involves using two forceps to pass a needle through portions of the tissue to be sutured, and then pass both ends of the suture through one trocar to the exterior of the body where a loose knot is formed. The loose knot is re-introduced into the body cavity through the trocar by a third instrument. Once the knot is at the appropriate location, the knot is tightened, resulting in a secure suture. This series of steps is repeated for each suture that is required. In addition to being very time-consuming, the loose knot frequently becomes tightened before it reaches the appropriate point. In addition, some instruments have pre-loaded suture material in a pre-formed loop which incorporates a slip knot. The loop is placed over a vessel or other structure where a suture is desired and the loop is tightened around the structure via the slip knot. This device and method suffer from several drawbacks. First, the instruments pre-loaded with suture material are expensive. Second, since the devices are pre-loaded with suture material, the surgeon's choice of suture material is limited. Finally, the device is effective only on structures that have a free end, and cannot be used in the place of sutures to join tissue together. Other methods that have been advanced to control bleeding and obviate the need for traditional sutures. These include the use of heated instruments to cut a structure and cauterize the structure in one step. Staples and clamps have also been used in the place of sutures in some cases.

The present disclosure is directed to a surgical instrument incorporating a modification allowing a surgeon to quickly and efficiently employ sutures during surgical procedures, such as laparoscopy. This modification allows the surgeon to simulate placing the surgical instrument in a position perpendicular to the suture thread, mimicking the commonly used technique for suture ligation.

### SUMMARY

The present disclosure is directed to a surgical instrument for assisting in the formation of a suture inside the body. The instrument comprises a modified surgical instrument of conventional design and operation (such as surgical forceps or laparoscopic graspers), the instrument further comprising at least one link, the link having a pair of moveable jaws operationally coupled to a first end and an alternating means to pivotally move the jaws adjacent to the second end. At least one link of the instrument is modified so that a knot assist assembly is operationally coupled to the at least one link. The knot assist assembly comprises a wire having a first end terminating in a semi-flexible tip and a second end operationally linked to an activating means for reversibly driving the flexible tip between a retraced position generally parallel to the long axis of the link and an extended position such that the tip prevents the suture thread from sliding off the instrument. It is preferred that the extended position be generally perpendicular to the long axis of the link, but other positions should be considered within the scope of the present disclosure so long as the function of preventing the suture thread from slipping off the instrument is retained. In the extended position the flexible tip is in such a position that the instrument functions as if the surgeon was placing the instrument at a perpendicular angle to the suture thread. As a result, the suture thread does not slip of the instrument, and sutures may be formed quickly and efficiently. Although the modified instrument of the present disclosure is particularly well suited to laparoscopic and endoscopic procedures, it may also be used in conventional surgical procedures where freedom of movement is restrained.

Therefore, an object of the disclosure is to provide a surgical instrument that will allow a surgeon, even one of limited experience, to utilize conventional sutures during a laparoscopic or other surgical procedure in an efficient manner.

It is another object of the disclosure to provide a surgical instrument that is reusable and can be subject to sterilization procedures.

Yet another object of the disclosure is to provide a surgical instrument which allows a surgeon the maximum amount of flexibility in choosing the type of suture material appropriate for a given procedure.

Yet another object of the disclosure to provide a method of using the surgical instrument to allow a surgeon to suture tissue, blood vessels or other structures in an efficient manner.

### BRIEF DESCRIPTION OF THE FIGURES

So that the features, advantages and objects of the disclosure will become clear, are attained and can be understood in detail, reference is made to the appended drawings, which are described briefly below. It is to be noted, however, that the appended figures that illustrate preferred embodiments of the disclosure, and therefore are not to be considered limiting in their scope.

FIG. 1 is a top perspective view of the surgical instrument of the present disclosure;

FIG. 2 is a side perspective view of the surgical instrument of the present disclosure;

FIG. 3 is a perspective view of the knot assist assembly;

FIG. 4 is an enlarged side view of the surgical instrument of the present disclosure showing the flexible tip of the knot assist assembly in detail in its extended position;

FIG. 5 is an enlarged side view of the surgical instrument of the present disclosure showing the flexible tip of the knot assist assembly in detail in its retracted position; and

FIG. 6 is a top perspective view of an alternate embodiment of the surgical instrument of the present disclosure.

### DETAILED DESCRIPTION

As discussed above, the use of sutures, while common in traditional, open surgical procedures, has not been extensively used in laparoscopic or endoscopic procedures. The surgical instrument of the present disclosure is based on a conventional surgical instrument, including but not limited to, forceps or laparoscopic graspers (collectively forceps). The instrument has been modified in such a way to incorporate a knot assist assembly to aid in suture ligation.

It is to be understood that although this disclosure will refer to surgical procedures, the term surgical procedures should include laparoscopic procedures, endoscopic procedures, as well as other surgical procedures where sutures may be employed. Furthermore, although the disclosure uses the term surgical instruments of conventional design, such as forceps or laparoscopic graspers, the modification should be understood so that it may be applied to any surgical instruments used to form sutures during surgical procedures. In addition, when referring to a “jaw” or “jaws” which are incorporated into the device, the term should be understood to include any structure that will allow a surgeon to grasp an object with the instrument. Also, the term “wire” when describing the knot assist assembly shall include any device or structure will allow the activating means to transmit force to the tip and drive the tip to the extended position, whether a wire in the conventional sense, a rod, or other device or structure. Finally, the term “extended” position when referring to the position of the tip means any position that is capable of preventing the suture thread from slipping off of the instrument so that a completed suture may be formed. In one embodiment, the extended position is described such that the tip is in a position generally perpendicular to the long axis of the link.

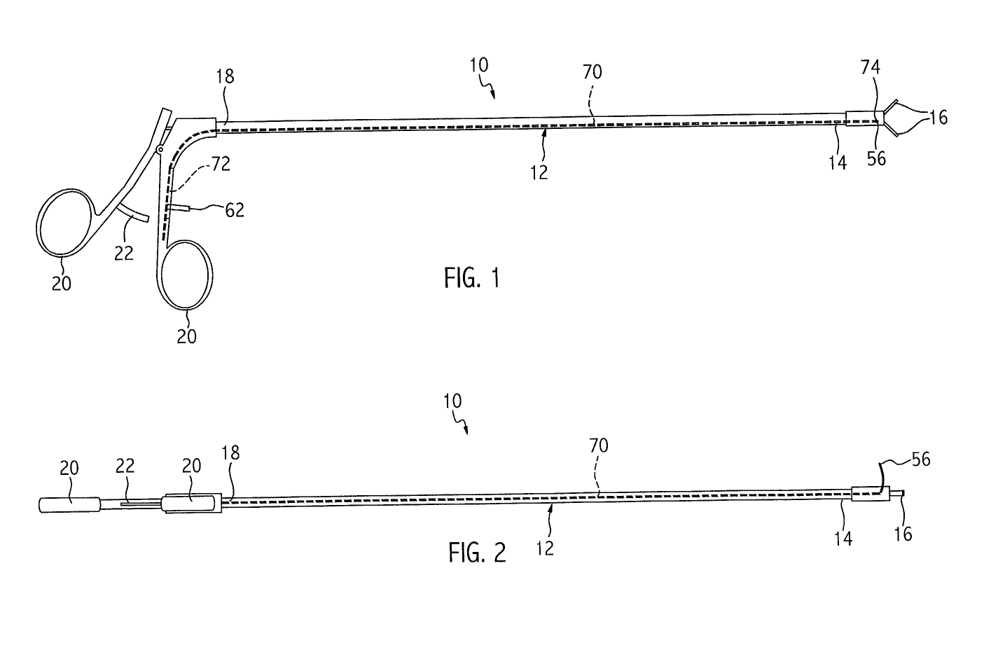

The instrument of the present disclosure is illustrated in FIGS. 1 and 2. The instrument 10 is a modified surgical instrument of conventional design (shown in FIGS. 1 and 2 as a modified laparoscopic grasper). The forceps 10 comprises at least one link 12 having a first end 14 adjacent to a pair of movable jaws 16. At least one of the jaws 16 is movable in relation to the other so that the jaws 16 can be alternated between an open position and a closed position (FIG. 1 illustrates the jaws 16 in an open position). The second end 18 of link 12 is operationally linked to an alternating means to alternate the jaws 16 between the open and closed position. In FIG. 1, the alternating means is illustrated as a pair of handles 20 operationally linked to a trigger 22. By manipulation of trigger 22, at least one of the jaws 16 is induced to move in relation to the other, thereby alternating the jaws 16 between the open and closed positions. Other alternating means would be obvious to one of ordinary skill in the art and should be considered within the scope of this disclosure.

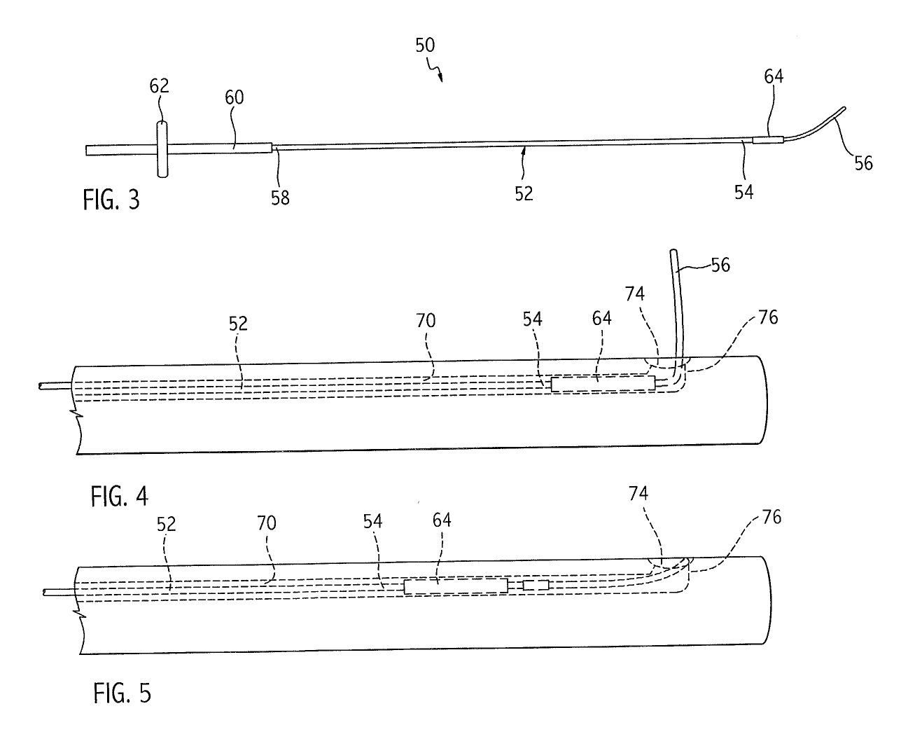

The knot assist assembly 50 (illustrated in greater detail in FIGS. 3-5) is operationally coupled to the instrument 10. Although the knot assist assembly 50 may be coupled to the exterior of the instrument 10, it is preferred that the knot assist assembly 50 be coupled to the interior of the instrument 10. The knot assist assembly 50 comprises a wire 52 having a first end 54 terminating with a semi-flexible tip 56 and a second end 58 operationally linked to an activating means for reversibly driving the tip 56 between a retracted position and an extended position. FIGS. 2 and 4 illustrate the tip 56 in the extended position. The activating means is illustrated in FIG. 3 as comprising a guide 60 operationally linked to second end 58 of wire 52, with guide 60 further comprising a trigger 62.

In a preferred embodiment, the knot assist assembly 50 is coupled to the interior of instrument 10 as illustrated in FIGS. 1 and 2. In this embodiment, an internal channel 70 is formed in the link 12 and at least one of the handles 20. The instrument 10 further comprises a trigger opening 72 in at least one of the handles 20 and a tip opening 74 on the link 12. The knot assembly 50 fits into the channel 70, with the guide 60 and wire 52 being at least partially enclosed by the channel 70. The trigger 62 extends through the trigger opening 72 and can be moved laterally along the long axis of the at least one handle 20 within the trigger opening 72 to reversibly drive the flexible tip 56 between the retracted and extended positions. In the embodiment shown in FIG. 1, when the trigger 62 is moved toward the jaws 16 to the limit of the trigger opening 72, the tip 56 is in the extended position and when the trigger 62 is moved away from the jaws 16 to the limit of the trigger opening 72 the tip 56 is in the retracted position. The tip 56 extends from the channel 70 through the tip opening 74 to assume the extended position. It is preferred that the tip opening 74 be positioned close to the jaws 16, but any placement of the tip opening 74 along link 12 should be considered encompassed by the present disclosure. FIGS. 4 and 5 illustrate a shoulder 76 at the end of channel 70 adjacent to jaws 16. The shoulder 76 assists the tip 56 in its exit from the channel 70 through the tip opening 74.

The wire 52 is designed so that the tip 56 has “memory,” that is the ability to adopt an extended position when driven out of channel 70 by manipulation of the trigger 62, but that allows the wire 52 and tip 56 to adopt the retracted position when desired. The knot assist assembly 50 may further comprise a tip guide 64 on wire 52 to aid the wire 50 in its traverse up and down channel 70. In addition, the tip 56 may be coupled to the wire 52 at the first end 54 via a flexible joint (not shown). The flexible joint aids the tip 56 in attaining the extended state. The flexible joint 66 and/or the “memory” of tip 56 when incorporated in the knot assist assembly each aid the tip in alternating between the extended and retracted positions.

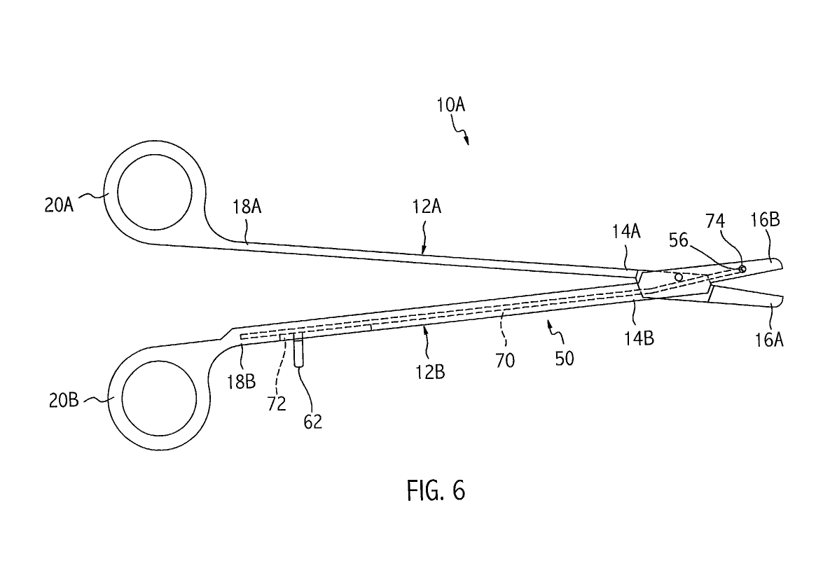

FIG. 6 illustrates an alternate embodiment of the instrument 10A. In this embodiment, the instrument 10 comprises a first link 12A and a second link 12B, the links being pivotally joined so that they are movable in relation to each other. Link 12A has a first end 14A and a second end 18A, while link 12B has a first end 14B and a second end 18B. The first ends 14A and 14B have a jaw 16A and 16B, respectively, adjacent to the first ends. The second ends 18A and 18B are adjacent to a means to pivotally move the first link 12A and second link 12B in relation to each other so that the jaws 16A and 16B can be manipulated between an open and closed position. FIG. 6 illustrates this means to pivotally move the links 12A and 12B as handles 20A and 20B. At least one of links 12A or 12B incorporates the knot assist assembly 50 as described above. It should be considered within the scope of this disclosure if both links 12A and 12B incorporate the knot assist assembly 50. As discussed above, the knot assist assembly 50 can be coupled to the exterior or interior of the instrument 10A, but it is preferred that the assembly 50 be coupled to the interior of the instrument 10A. At least one link 12A or 12B and at least one of the handles 20A or 20B contains an internal channel 70A to receive the knot assist assembly 50. The instrument 10A further comprises a trigger opening 72 in at least one of the handles 20A or 20B and a tip opening 74A on at least one of link 12A or 12B. The knot assembly 50 fits into the channel 70A, with the guide 60 and wire 52 being at least partially enclosed by the channel 70A. The trigger 62 extends through the trigger opening 72A and can be moved laterally along the long axis of the at least one handle 20A within the trigger opening 72 to reversibly drive the flexible tip 56 between the retracted and extended positions. In the embodiment shown in FIG. 6, when the trigger 62 is moved toward the jaws 16A and 16B to the limit of the trigger opening 72A, the tip 56 is in the extended position and when the trigger 62 is moved away from the jaws 16A and 16B to the limit of the trigger opening 72A the tip 56 is in the retracted position. The tip 56 extends from the channel 70A through the tip opening 74A to assume the extended position. It is preferred that the tip opening 74A be positioned close to the jaws 16A and 16B, but any placement of the tip opening 74A along at least one of link 12A or 12B should be considered encompassed by the present disclosure. A shoulder 76A may also be incorporated into the end of channel 70A adjacent to jaws 16A and 16B to assist the tip 56 in its exit from the channel 70A through the tip opening 74A.

The instrument 10 (including all component parts, including, but not limited to the knot assist assembly 50) may be manufactured from a variety of materials commonly used for such instruments. The selection of the appropriate material is well within the ordinary skill of one in the medical device field. A preferred material for the manufacture of the instrument 10 is stainless steel.

The following provides and example of how one embodiment of the instrument 10 might be used during a laparoscopic procedure. This example should not be interpreted as limiting the use of the instrument 10 solely to laparoscopic procedures, but as an illustration of its operation. In operation, the instrument 10 is placed through a trocar tube so that at least a portion of the link 12 is within a body cavity, such as the abdomen, of a patient. When it is desired to incorporate a suture into a tissue during the procedure, the surgeon may use the instrument 10 to pass a needle through the tissue to be sutured by manipulating the jaws 16 to grasp a needle attached to a suture thread (not shown) and penetrating the tissue to be sutured. Once the needle has penetrated the tissues to be sutured, the surgeon manipulates the jaws 16 to release the needle and places the link 12 adjacent to the suture thread at a desired location to initiate the formation of the suture. As discussed above, due to spatial constraints, the link 12 must be placed horizontal to the suture thread. The surgeon manipulates the instrument 10 so that the suture thread is wrapped at least once around the link 12. At this point, the surgeon pushes the trigger 62 toward the jaws 16 to engage the guide 60 and drive the tip 56 out the tip opening 74 so that the tip 56 is in its extended position. So placed, the tip 56 prevents the suture thread from sliding of the end of the link 12. Once the tip is in the extended position, the surgeon again manipulates the jaws 16 through the use of trigger 22 so that the jaws are in the open position. The surgeon then places the jaws in a position to grasp the suture thread near the needle and closes the jaws 16 around the suture thread through the use of trigger 22. The surgeon then pushes the trigger 62 away from the jaws 16 so that the tip 56 is driven to its retracted position and manipulates the instrument 10 so that the suture thread in jaws 16 passes through the now looped suture thread on link 12. In this manner the suture is ligated (i.e., a suture knot is formed). The procedure can be repeated until the tissue is completely sutured.

The method described above has many advantages over current methods for forming sutures in laparoscopic procedures. First, the suture is formed completely inside the body cavity of a patient without the need for removing the suture through a trocar to form the suture knot, thus saving time and allowing for more accurate placement of the suture. Second, since the instrument of the current disclosure is not supplied with a particular type of suture, the surgeon is given more flexibility in choosing the type of suture appropriate for the procedure at hand. Finally, the method of forming sutures using the instrument of the present disclosure is simple to use requiring minimal retraining for the surgeon to become proficient in using the instrument. Finally, the suture procedure can be performed quickly and accurately, allowing the surgeon to respond to unanticipated events that would before require the laparoscopic procedure be terminated and conventional surgery initiated.

1. A modified surgical instrument of conventional design for assisting in suture ligation, the instrument comprising at least one knot assist assembly operationally coupled to the instrument, the assembly comprising a wire having a first end terminating with a tip and a second end operationally linked to an activating means for reversibly driving the tip between a retracted position and an extended position.

2. The surgical instrument of claim 1 further comprising at least one link operationally coupled to at least one jaw, the at least one jaw being capable of alternating between an open or a closed position, and an alternating means for alternating the at least one jaw between the open and closed positions.

3. The surgical instrument of claim 2 where the activating means further comprises a guide and a trigger extending from the guide, the guide being operationally linked to the second end of the wire such that pushing the lever toward the jaws causes the tip to be in one of either the retracted or extended positions, and pushing the lever away from the jaws causes the tip to be in the other of either the retracted or extended positions.

4. The surgical instrument of claim 3 where pushing the trigger toward the jaws causes the tip to be in the extended position and pushing the trigger away from the jaws causes the tip to be in the retracted position.

5. The surgical instrument of claim 1 where the assembly further comprises a tip guide positioned on the wire between about the tip and the guide.

6. The surgical instrument of claim 1 where the tip of the wire incorporates memory flexibility through a flexible joint or a slight bend in the tip or the combination of a flexible joint and a slight bend in the tip.

7. The surgical instrument of claim 1 further comprising an internal channel for receiving the knot assist assembly, such that the wire and the activating means are contained at least partially within the internal channel, a trigger opening on the instrument such that a portion of the activating means extends through the trigger opening, and a tip opening on the instrument.

8. The surgical instrument of claim 7 further comprising at least one link operationally coupled to at least one jaw, the at least one jaw being capable of alternating between an open or a closed position, and an alternating means for alternating the at least one jaw between the open and closed positions.

9. The surgical instrument of claim 8 where the activating means further comprises a guide and a trigger extending from the guide, the guide being operationally linked to the second end of the wire such that pushing the lever toward the jaws to the limit of the trigger opening causes the tip to be in one of either the retracted or extended positions, and pushing the lever away from the jaws to the limit of the trigger openings causes the tip to be in the other of either the retracted or extended positions.

10. The surgical instrument of claim 9 where pushing the trigger toward the jaws to the limit of the trigger openings causes the tip exit the channel through the tip opening to be in the extended position and pushing the trigger away from the jaws to the limit of the trigger openings causes the tip to reenter the channel through the tip opening to be in the retracted position.

11. The surgical instrument of claim 7 where the assembly further comprises a tip guide positioned on the wire between about the tip and the guide.

12. The surgical instrument of claim 7 where the tip of the wire incorporates memory flexibility through a flexible joint or a slight bend in the tip or the combination of a flexible joint and a slight bend in the tip.

13. The surgical instrument of claim 7 where the guide wire element is contained substantially within the internal channel.

14. The surgical instrument of claim 7 further comprising a shoulder at the end of the internal channel adjacent to the jaws.

15. The surgical instrument of claim 1 further comprising a first link and a second link, the first and second links each having a first end adjacent to a jaw, the first and second links being pivotally moveable in relation to each other so that the jaws may alternate between an open and closed position, and an alternating means for alternating the jaws between the open and closed positions.

16. The surgical instrument of claim 15 where the activating means further comprises a guide and a trigger extending from the guide, the guide being operationally linked to the second end of the wire such that pushing the lever toward the jaws causes the tip to be in one of either the retracted or extended positions, and pushing the lever away from the jaws causes the tip to be in the other of either the retracted or extended positions.

17. The surgical instrument of claim 15 further comprising an internal channel for receiving the knot assist assembly, such that the wire and the activating means are contained at least partially within the internal channel, a trigger opening on the instrument such that a portion of the activating means extends through the trigger opening, and a tip opening on the at least one of the first or second links so that the tip can exit and enter the channel.

18. The surgical instrument of claim 17 where the activating means further comprises a guide and a trigger extending from the guide, the guide being operationally linked to the second end of the wire such that pushing the lever toward the jaws to the limit of the trigger opening causes the tip to be in one of either the retracted or extended positions, and pushing the lever away from the jaws to the limit of the trigger openings causes the tip to be in the other of either the retracted or extended positions.

19. The surgical instrument of claim 18 where pushing the trigger toward the jaws to the limit of the trigger openings causes the tip exit the channel through the tip opening to be in the extended position and pushing the trigger away from the jaws to the limit of the trigger openings causes the tip to reenter the channel through the tip opening to be in the retracted position.

20. A method of suture ligation, the method comprising the steps of:
a. placing a surgical instrument comprising a knot assist assembly into a body cavity of a patient;
b. using the surgical instrument to pass a needle in combination with a suture thread through a tissue to be sutured;
c. releasing the needle and placing the at least one link of the instrument adjacent to the suture thread at a location where the suture is desired and manipulating the surgical instrument so that the suture thread wraps at least once around the at least one link;
d. pushing the trigger on the activating means such that the tip assumes the extended position;
e. grasping the suture thread with the jaws of the instrument;
f. pushing the trigger on the activating means such that the tip assumes the retracted position; and
g. manipulating the instrument so that the jaws pass through the looped suture thread to form a knot in the suture thread.
